(19) United States
(12) Patent Application Publication
(10) Pub. No.: US 2024/0018090 A1
(43) Pub. Date: Jan. 18, 2024

(54) Processes for the Preparation of Fenfluramine

(71) Applicant: Apotex Inc., Toronto (CA)

(72) Inventors: Avedis Karadeolian, Brantford (CA); Fabio E. S. Souza, Brantford (CA); Allan W. Rey, Brantford (CA)

(21) Appl. No.: 18/228,086

(22) Filed: Jul. 31, 2023

Related U.S. Application Data
(60) Provisional application No. 63/394,024, filed on Aug. 1, 2022.

(51) Int. Cl. C07C 209/74; C07C 209/90; C07C 211/15; B01J 23/44; B01J 21/18

(52) U.S. Cl. CPC C07C 209/74; C07C 209/90; C07C 211/15; B01J 23/44; B01J 21/18

(57) ABSTRACT

The present invention provides processes for the preparation of fenfluramine and salts thereof, including reductive amination of 3-(trifluoromethyl) phenylacetone, as well as products prepared by such processes.

### CROSS-REFERENCE TO RELATED APPLICATION

This application claims priority to U.S. Provisional Patent Application No. 63/394,024 filed Aug. 1, 2022, the disclosure of which is hereby incorporated by reference in its entirety.

### BACKGROUND OF THE INVENTION

### Field of the Invention

The present invention relates to processes for the preparation of fenfluramine, and salts thereof, as well as to the products of such processes.

### Description of Related Art

Fenfluramine (1), or N-ethyl-α-methyl-3-(trifluoromethyl) phenethylamine is the active pharmaceutical ingredient (API), in the form of the hydrochloride salt, in FINTEPLA®, a medication indicated for the treatment of seizures associated with Dravet syndrome in patients 2 years of age and older.

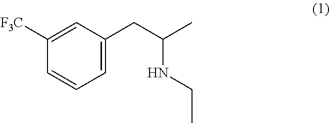 [SMILES: CCNC(C)Cc1cccc(C(F)(F)F)c1]

A process for the preparation of a series of 1-(trifluoromethylphenyl)-2-lower-alkylaminopropane compounds, which includes fenfluramine, is disclosed in U.S. Pat. No. 3,198,833 A, which is depicted in Scheme 1. Amine (B) is treated with acetic anhydride to afford acetylated intermediate (A), which undergoes reduction with lithium aluminum hydride to afford fenfluramine (1), and subsequently, the hydrochloride salt thereof.

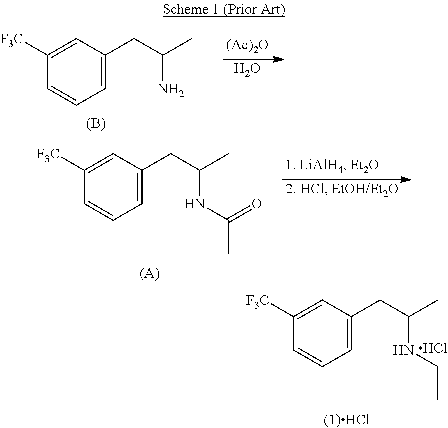

A second synthetic method for the preparation of fenfluramine is described in EP 3 170 807 B1. In this process, which is exemplified in Scheme 2, ketone (D) is reacted with ethylamine hydrochloride to afford enamine (C), which undergoes reduction in the presence of sodium borohydride to afford fenfluramine (1), that is then further converted to the hydrochloride salt.

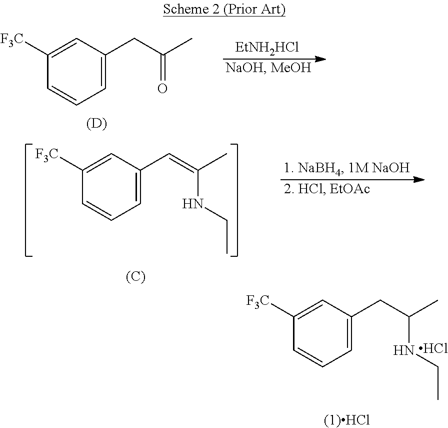

A number of other procedures reported for the preparation of fenfluramine, including, Su et al. Gaodeng Xuexiao Huaxue, 1988, 9 (2), 134, Ji et al. Shenyang Yaoxueyuan Xuebao, 1994, 11 (2), 116, Xu et al. Shenyang Yaoke Daxue Xuebao, 1995, 12 (3), 171, Willcox et al. Science, 2016, 354 (6314), 851, and WO 2017/112702 A1 follow the same basic synthetic approach, comprising reductive amination of ketone (D) with ethylamine and a metal hydride reductant.

In WO 2021/117057 A1, as shown in Scheme 3, ketone (D) is condensed with ethylamine, followed by reduction of the obtained compound in the presence of hydrogen gas and a Pd/C catalyst, to afford fenfluramine (1) and subsequently the hydrochloride salt thereof. Similar procedures are reported in, for example, DD108971, HU204497, and Chen et al. Huagong Shikan, 2002, 16 (7), 33.

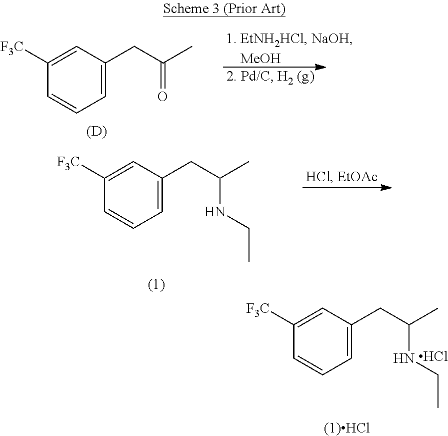

The existing processes for the preparation of fenfluramine suffer from various problems including generation of impurities, including the alcohol derived from competitive reduction of ketone (D), use of flammable hydrogen gas or metal hydride reducing agents, and/or use of toxic organic solvents such as methanol.

Owing to the drawbacks of the existing processes, there remains a need for improved processes for the preparation of fenfluramine (1), that are more amenable to scale-up and use on a commercial scale.

### SUMMARY OF THE INVENTION

The present invention provides improved processes for the preparation of fenfluramine (1), or a salt thereof, as depicted in Scheme 4.

As shown in Scheme 4, in the processes of the present invention, fenfluramine (1), or a salt thereof, may be prepared by a process comprising reacting 3-(trifluoromethyl)phenylacetone with ethylamine, or a salt thereof, in the presence of an aqueous mixture comprising a heterogenous light platinum-group metal catalyst, and a hydrosilane, preferably in the presence of a surfactant.

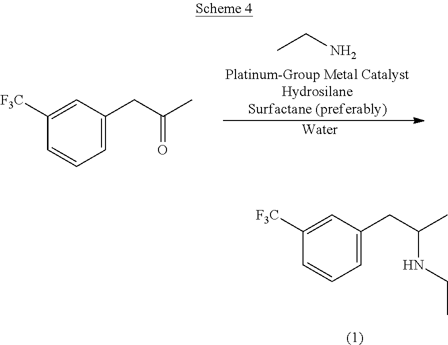

The present invention provides processes which can be performed in water and which avoid the use of reactive metal hydrides. Further embodiments are provided for the preparation of fenfluramine, or a salt thereof, having high purity in an efficient manner using common reagents.

Accordingly, in a first aspect of the present invention, there is provided a process for the preparation of fenfluramine (1), or a salt thereof, the process comprising reacting 3-(trifluoromethyl)phenylacetone with ethylamine, or a salt thereof, in the presence of an aqueous mixture comprising a heterogenous light platinum-group metal catalyst, and a hydrosilane. In a preferred embodiment of the first aspect, the aqueous mixture further comprises a surfactant. Preferably, the catalyst is selected from the group consisting of palladium and ruthenium, more preferably the catalyst is palladium supported on carbon (Pd/C). In a further preferred embodiment of the first aspect, the amount of palladium with respect to 3-(trifluoromethyl)phenylacetone is in the range of about 0.2 mol % and about 1 mol %. In another preferred embodiment of the first aspect, the hydrosilane is selected from the group consisting of triethylsilane, phenylsilane, phenylmethylsilane, diphenylsilane, and polylmethylhydrogensiloxane, preferably the hydrosilane is triethylsilane. In a further preferred embodiment of the first aspect, the surfactant is a non-ionic surfactant or an anionic surfactant. Preferably, the non-ionic surfactant is selected from the group consisting of ethoxylates and fatty acid esters of polyhydroxy compounds, and wherein the anionic surfactant is selected from the group consisting of carboxylic acids, carboxylate salts, sulfate salts, and phosphate salts, more preferably, the surfactant is stearic acid or a salt thereof. In another preferred embodiment of the first aspect, the amount of surfactant with respect to 3-(trifluoromethyl)phenylacetone is in the range of about 1 wt % and about 5 wt %. In a further preferred embodiment of the first aspect, the reaction is conducted in water that is substantially free of organic solvents. In another preferred embodiment of the first aspect, the reaction is conducted in a sealed vessel at a temperature in the range of about 20° C. and about 100° C. Preferably, the fenfluramine is further converted to a pharmaceutically acceptable salt thereof, further preferably the salt is fenfluramine hydrochloride.

In a second aspect of the present invention, there is provided fenfluramine, or a salt thereof, that is produced by any one of the processes of the first aspect of the invention. In a preferred embodiment of the second aspect the fenfluramine or salt thereof has no more than about 0.2 area %, further preferably no more than about 0.1 area %, and more preferably, no more than about 0.05 area % 1-[3-(trifluoromethyl)phenyl]-2-propanol as determined by HPLC analysis. In a further preferred embodiment of the second aspect, the fenfluramine, or salt thereof, has no more than about 10 ppm of catalyst metal impurities with respect to fenfluramine.

In a third aspect of the present invention, there is provided a pharmaceutical composition comprising the fenfluramine, or a salt thereof, according to the second aspect of the invention, and one or more pharmaceutically acceptable excipients. In a preferred embodiment of the third aspect, the pharmaceutical composition is an oral solution.

### DESCRIPTION OF THE INVENTION

The processes of the present invention provide fenfluramine (1), or a salt thereof, from readily available materials in a concise, practical, and industrially applicable manner.

As used herein, the term “wt %” (% weight/weight) refers to the ratio of the weight of a subject component to the weight of the subject mixture, using the same weight unit, expressed as a percentage. For example, with respect to the amount of surfactant, wt % refers to: (weight of the surfactant in g/(weight of surfactant in g+weight of 3-(trifluoromethyl)phenylacetone) in g)×100.

As used herein, the term “ppm” (parts per million) when used in reference to purity, refers to the ratio of the weight of a subject component to the weight of another component, using the same weight unit, multiplied by one million. For example, with respect to the amount of heavy-metal impurities, ppm refers to: (weight heavy metal in mg/weight fenfluramine in mg)×1,000,000.

As used herein, “room temperature” generally refers to a temperature of 20-25° C.

As used herein, the term “about” means “close to”, and that variation from the exact value that follows the term is within amounts that a person of skill in the art would understand to be reasonable. For example, when the term “about” is used with respect to temperature, a variation of ±5° C. is generally acceptable when carrying out the processes of the present invention. When used with respect to mole equivalents, a variation of ±0.1 moles is generally acceptable.

In one embodiment of the present invention, fenfluramine (1), or a salt thereof, may be prepared by the process as set out in Scheme 4. Exemplary reagents and conditions for these processes are described herein.

In one embodiment of the present invention, there is provided a process for the preparation of fenfluramine (1):

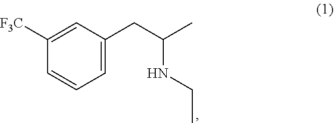 [SMILES: CCNC(C)Cc1cccc(C(F)(F)F)c1]

the process comprising reacting 3-(trifluoromethyl)phenylacetone with ethylamine, or a salt thereof, in the presence of an aqueous mixture comprising a heterogenous light platinum-group metal catalyst, and a hydrosilane.

The heterogenous light platinum-group catalyst is selected from the group consisting of ruthenium, rhodium, and palladium. Preferably, the catalyst is ruthenium or palladium, most preferably palladium. The catalyst may be finely dispersed solids or adsorbed on an inert support such as carbon or alumina and may be wet or dry. The catalyst may be 10 wt % palladium on carbon (dry basis) and from about 50 wt % to 70 wt % (wet support), preferably it is 10 wt % palladium on carbon (dry basis), 66 wt % (wet support). The catalyst loading may be from about 0.2 mol % to about 1 mol % palladium with respect to the amount of 3-(trifluoromethyl)phenylacetone, preferably about 0.4 mol %.

The hydrosilane is an organosilane reducing agent bearing a hydride. Preferably, the hydrosilane is selected from the group consisting of triethylsilane, phenylsilane, phenylmethylsilane, diphenylsilane, and polymethylhydrogensiloxane, most preferably it is triethylsilane.

The reaction is conducted in an aqueous environment. Preferably, water is the reaction medium and the reaction mixture is substantially free of other solvents, including organic solvents.

The reaction is preferably conducted in the presence of a surfactant. The role of the surfactant is to, for example, increase the miscibility of the reactants in the system. Preferably, the surfactant is a non-ionic surfactant such as an ethoxylate (e.g. nonoxynols, Triton X-100, ‘designer’ surfactant TPGS-750-M) or a fatty acid ester of a polyhydroxy compound (e.g. Spans or Tweens), or it is an anionic surfactant such as a carboxylic acid (e.g. stearic acid), a carboxylate salt (e.g. magnesium stearate), a sulfate salt (e.g. sodium dodecyl sulfate), or a phosphate salt (e.g. alkyl phosphates). Most preferably, the surfactant is stearic acid or a salt thereof. The amount of surfactant with respect to 3-(trifluoromethyl)phenylacetone is preferably at least about 1 wt % and is preferably in the range of about 1 wt % and about 5 wt %.

The reaction may be conducted at any suitable temperature, and is preferably conducted at a temperature in the range of about 20° C. to about 100° C. or the boiling point of the reaction mixture. Preferably, the reaction is conducted in a sealed vessel and the suitable temperature is in the range of about 50° C. to about 70° C.

The fenfluramine may be further converted to a pharmaceutically acceptable salt thereof, preferably the hydrochloride salt. Further purification of the fenfluramine or a salt thereof may be conducted, if necessary, by crystallization from a suitable solvent such as an ester or alcohol.

In a second embodiment, the present invention provides fenfluramine, or salt thereof, having high purity that may be expressed as a purity as measured by the area percent (area %) of the respective peaks attributed to fenfluramine or its impurities by a high-performance liquid chromatography (HPLC) method described herein, or any other suitable method. One impurity that has been identified in processes for the preparation of fenfluramine is the associated reduced alcohol impurity 1-[3-(trifluoromethyl)phenyl]-2-propanol. Embodiments of the present invention provide fenfluramine, or a salt thereof, having no more than about 0.2 area % 1-[3-(trifluoromethyl)phenyl]-2-propanol as determined by HPLC analysis. Preferably, fenfluramine, or a salt thereof, is provided having no more than about 0.1 area % 1-[3-(trifluoromethyl)phenyl]-2-propanol as determined by HPLC analysis. More preferably, fenfluramine, or a salt thereof, is provided having no more than about 0.05 area % 1-[3-(trifluoromethyl)phenyl]-2-propanol as determined by HPLC analysis. Furthermore, embodiments of the present invention provide fenfluramine, or a salt thereof, having low levels of residual catalyst metal impurities as measured by, for example, standard ICP-MS methods such as US Pharmacopeia (USP) <232> (Elemental Impurities-Limits) and USP <233> (Elemental Impurities-Procedures). Preferably, fenfluramine, or a salt thereof, is provided having no more than about 10 ppm, no more than about 5 ppm, and no more than about 2 ppm residual catalyst metal impurities (i.e. palladium, ruthenium, and rhodium).

In a third embodiment of the invention, there is provided a pharmaceutical composition comprising fenfluramine, or a pharmaceutically acceptable salt thereof, prepared by the processes described herein, with one or more pharmaceutically acceptable excipients. Preferably, the pharmaceutical composition is an oral solution. Preferably, the pharmaceutical composition provides 2.2 mg/mL fenfluramine, equivalent to the concentration found in FINTEPLA® drug products.

Suitable pharmaceutically acceptable excipients are preferably inert with respect to the fenfluramine and salts thereof of the present invention, and may include, for example, one or more excipients selected from solvents, such as water; colorants such as Colorcon® colorants; flavorants such as grape, cherry, orange, peppermint, menthol, and vanilla flavors; sweeteners such as sucrose, liquid glucose, sorbitol, dextrose, isomalt, liquid maltitol, aspartame, acesulfame K, and lactose; preservatives such as methyl, ethyl, propyl and butyl parabens. Other excipients including pH modifying agents such as citrate and phosphate buffers, viscosity modifiers such as water-soluble cellulose derivatives, and anti-oxidants may be added as required. Other suitable excipients and the preparation of liquid oral dosage forms are well known to person of skill in the art, and is described generally, for example, in Remington The Science and Practice of Pharmacy 21st Edition (Lippincott Williams & Wilkins: Philadelphia; 2006; Chapter 39).

### EXAMPLES

The following examples are illustrative of some of the embodiments of the invention described herein. It will be apparent to the person skilled in the art that various alterations to the described processes in respect of the reactants, reagents, and conditions may be made when using the processes of the present invention without departing from the scope or intent thereof. All materials and reagents, including 3-(trifluoromethyl)phenylacetone were commercially available and can furthermore be prepared by published literature methods.

### Analysis Method for Determining the Chromatographic Purity of Fenfluramine (1)

The method shown in Table 1 was used to determine the chromatographic purity of samples of fenfluramine (1) as provided in the examples that follow.

TABLE 1
HPLC method for the determination of chromatographic
purity of fenfluramine (1).

| Instrument | Waters ® 2695 | | |
|---|---|---|---|
| Column | Hypersil ™ BDS C18 (5 microns, 0.46 cm i.d. × 25 cm) | | |
| Column Temp. | 20-25° C. | | |
| Sample temp. | 20-25° C. | | |
| Mobile phase | Mobile Phase A: 0.1M K₂HPO₄, pH 7.0 with H₃PO₄ | | |
| | Mobile Phase B: Acetonitrile | | |
| | Time | % Solution A | % Solution B |
| Gradient | 0 | 65 | 35 |
| Program | 30 | 65 | 35 |

| | |
|---|---|
| Flow rate | 1.2 mL/minute |
| Injection | 10 μL |
| volume | |
| Detector | Waters ® W2996 PDA detector, set to 264 nm detection |
| Run time | 30 minutes |
| Sample prep. | Dissolve sample in 65:35 A:B mobile phase mixture |
| Retention | Fenfluramine: 4.747 (RRT 1.00) |
| times | 1-[3-(trifluoromethyl)phenyl]-2-propanol RRT 3.66 |
| | 3-(trifluoromethyl)phenylacetone RRT 5.10 |

### Example 1: Preparation of Fenfluramine Hydrochloride (1)·HCl

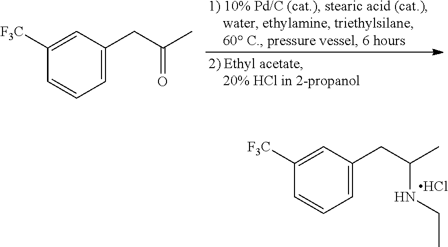

A pressure vessel containing a magnetic stir bar was charged with 10% Pd/C, 66% wet, (0.19 g, 0.4 mol %), stearic acid (0.06 g, 2 wt %), water (30 mL), 3-(trifluoromethyl)phenylacetone (2.4 mL, 3.0 g, 14.8 mmol), 70% ethylamine in water (3.0 mL, 2.4 g, 2.5 eq.), and triethylsilane (4.2 mL, 3.12 g, 1.8 eq.), and the vessel was sealed and heated to an external temperature of 60° C. for 6 hours. After the allotted time, HPLC analysis of the reaction mixture showed all the starting material was consumed (97.03 a % fenfluramine, 0.22 a % 1-[3-(trifluoromethyl)phenyl]-2-propanol). The reaction mixture was filtered through diatomaceous earth and washed with ethyl acetate. The aqueous phase was acidified to pH 1-2 and separated from the organic phase. The aqueous phase was washed with ethyl acetate, then made basic with 50% NaOH solution to pH 12-14. The aqueous phase was extracted with ethyl acetate (2×20 mL) and the organic phases were combined and filtered through anhydrous sodium sulfate. The filtrate was concentrated to afford fenfluramine free base as a clear, colourless liquid (2.4 g, 70% yield, 98.33 a % purity by HPLC).

The fenfluramine free base (2.4 g) was dissolved in ethyl acetate (48 mL) and charged with excess 20% HCl in isopropanol. A thick white suspension formed which was stirred at room temperature for 2 hours. The solid was collected by filtration and washed with ethyl acetate prior to drying in vacuo to furnish fenfluramine hydrochloride as a crystalline white solid (2.0 g, 50% yield from 3-(trifluoromethyl)phenylacetone) having 99.59 area % purity by HPLC and undetectable levels of 1-[3-(trifluoromethyl)phenyl]-2-propanol.

¹H-NMR of fenfluramine hydrochloride (DMSO-d₆, 300 MHz, ppm): δ 9.25 (broad s, 2H), 7.70-7.50 (m, 4H), 3.46 (broad s, 1H), 3.41 (dd, J=13.0, 3.0 Hz, 1H), 3.01 (broad s, 2H), 2.80 (dd, J=13.0, 10.0 Hz, 1H), 1.27 (t, J=7.2 Hz, 3H), 1.11 (d, J=6.5 Hz, 3H).

### Example 2: Preparation of Fenfluramine (1)

A small sealed vial containing 10% Pd/C, 66% wet (0.8 mol %), surfactant (0.1 g), water (5 mL), 3-(trifluoromethyl)phenylacetone (0.5 g), 70% ethylamine in water (0.5 mL), and triethylsilane (0.71 mL) was heated to an external temperature of 60° C. for 20 hours. The reaction mixture was sampled as indicated for HPLC analysis and the area % ratio of fenfluramine (1): 3-(trifluoromethyl)phenylacetone (′ketone) was used to determine the percent conversion. The reaction parameters and results of the HPLC analysis of the reaction mixtures are shown in Table 2.

TABLE 2
Reaction Parameters and HPLC Analysis of Examples 2a-2d

| | | Area % (1):ketone | Area % (1):ketone |
|---|---|---|---|
| | | after | after |
| Example | Surfactant | 3-4 h | overnight |
| 2a | TPGS-750-M¹ | 94:6 | 98:2 |
| 2b | Stearic acid | 94:6 | 98:2 |
| 2c | Span ® 80 | 69:31 | 78:22 |
| 2d | Tween ® 60 | 82:18 | 88:12 |

¹See Lipshutz B H, Ghorai S. 2012. “Designer”-Surfactant-Enabled Cross-Couplings in Water at Room Temperature. Aldrichimica Acta. 45(1): 3-16.

What is claimed is:

1. A process for the preparation of fenfluramine: 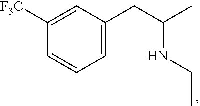 [SMILES: CCNC(C)Cc1cccc(C(F)(F)F)c1]
or a salt thereof, the process comprising reacting 3-(trifluoromethyl)phenylacetone with ethylamine, or a salt thereof, in the presence of an aqueous mixture comprising a heterogenous light platinum-group metal catalyst, and a hydrosilane.

2. The process of claim 1, wherein the aqueous mixture further comprises a surfactant.

3. The process of claim 1, wherein the catalyst is selected from the group consisting of palladium and ruthenium.

4. The process of claim 3, wherein the catalyst is palladium supported on carbon (Pd/C).

5. The process of claim 4, wherein the amount of palladium with respect to 3-(trifluoromethyl)phenylacetone ranges from about 0.2 mol % and about 1 mol %.

6. The process of claim 1, wherein the hydrosilane is selected from the group consisting of triethylsilane, phenylsilane, phenylmethyl silane, diphenylsilane, and polylmethylhydrogensiloxane.

7. The process of claim 6, wherein the hydrosilane is triethylsilane.

8. The process of claim 2, wherein the surfactant is a non-ionic surfactant or an anionic surfactant.

9. The process of claim 8, wherein the non-ionic surfactant is selected from the group consisting of ethoxylates and fatty acid esters of polyhydroxy compounds and wherein the anionic surfactant is selected from the group consisting of carboxylic acids, carboxylate salts, sulfate salts and phosphate salts.

10. The process of claim 9, wherein the surfactant is stearic acid or a salt thereof.

11. The process of claim 8, wherein the amount of surfactant with respect to 3-(trifluoromethyl)phenylacetone ranges from about 1 wt % and about 5 wt %.

12. The process of claim 1, wherein the reaction is conducted in water that is substantially free of organic solvents.

13. The process of claim 1, wherein the reaction is conducted in a sealed vessel at a temperature that ranges from about 20° C. to about 100° C.

14. The process of claim 1, wherein the fenfluramine is further converted to a pharmaceutically acceptable salt thereof.

15. The process of claim 14, wherein the salt is fenfluramine hydrochloride.

16. A fenfluramine, or a salt thereof, that is produced by the process of claim 1.

17. The fenfluramine or salt thereof of claim 16, having no more than about 0.2 area % 1-[3-(trifluoromethyl)phenyl]-2-propanol as determined by HPLC analysis.

18. The fenfluramine or salt thereof of claim 16, having no more than about 0.1 area % 1-[3-(trifluoromethyl)phenyl]-2-propanol as determined by HPLC analysis.

19. The fenfluramine or salt thereof of claim 16, having no more than about 0.05 area % 1-[3-(trifluoromethyl)phenyl]-2-propanol as determined by HPLC analysis.

20. The fenfluramine, or salt thereof, of claim 16, having no more than about 10 ppm of catalyst metal impurities with respect to fenfluramine.

21. A pharmaceutical composition comprising the fenfluramine, or a salt thereof, according to claim 16, and one or more pharmaceutically acceptable excipients.

22. The pharmaceutical composition of claim 21, wherein the pharmaceutical composition is an oral solution.
